(19) United States
(12) Patent Application Publication
(10) Pub. No.: US 2021/0338625 A1
(43) Pub. Date: Nov. 4, 2021

(54) USE OF POLYOL FATTY ESTER COMPOSITIONS AND PRODUCTS FOR MINIMIZING EXPOSURE TO A NOXIOUS SUBSTANCE

(71) Applicant: Mark KWIECINSKI, Ottawa, Ontario (CA)

(72) Inventor: Mark KWIECINSKI, Ottawa, Ontario (CA)

(21) Appl. No.: 17/311,141

(22) PCT Filed: Dec. 4, 2019
(86) PCT No.: PCT/CA2019/051748

§ 371 Date: Jun. 4, 2021

Related U.S. Application Data
(60) Provisional application No. 62/774,975, filed on Dec. 4, 2018.

(51) Int. Cl. A61K 31/232; A61K 31/23; A61K 31/231; A61K 9/00; A61K 9/12; A61K 9/70

(52) U.S. Cl. CPC A61K 31/232; A61K 31/23; A61K 9/7007; A61K 9/0014; A61K 9/12; A61K 31/231

(57) ABSTRACT

Method for minimizing exposure to a noxious substance, the method comprising the step of administering a spray composition to a surface or applying a wipe to a surface contaminated by the noxious substance, wherein the composition in the spray or impregnated into the wipe comprises a polyol fatty ester.

### FIELD OF THE INVENTION

The present invention pertains to the field of polyol fatty ester compositions and products and in particular to their use for minimizing exposure to noxious substances.

### BACKGROUND

First responders and front-line health and community workers are often exposed to dangerous substances during the course of carrying out their daily jobs. Although protective clothing and gear can reduce the overall chemical exposure, there are instances when it is impossible to avoid accidental or ongoing contact with chemicals or substances that can cause harm.

Examples of substances that can cause harm to first responders upon accidental contact include street drugs such as fentanyl or its analogues. Fentanyl is a powerful synthetic opioid pain medication administered to patients to prevent pain following surgery, for the management of chronic pain, and to produce sedation during medical procedures. Although similar in effect to morphine and heroin, fentanyl is 50 to 100 times more potent. Carfentanil is an analog of fentanyl that is 100 times more powerful than fentanyl, 5,000 times more powerful than heroin, and 10,000 times more powerful than morphine. Since fentanyl and carfentanil are much stronger than most other opioids, they can be very dangerous if misused, and even a small amount can lead to overdose and death.

Fentanyl and carfentanil are also sold as street drugs. Street fentanyl can be recovered from prescription transdermal patches or it can be produced illegally in drug labs. Street fentanyl and carfentanil in powder form may be swallowed, smoked, snorted or injected, or can cut into (mixed with) street drugs such as heroin or cocaine. Many overdoses have occurred because people did not know that the street drug they were taking was contaminated with fentanyl or carfentanil. In addition, it is common for first responders rushing to help overdose victims to get overdosed themselves due to secondary exposure with fentanyl or carfentanil from the body of the original overdose victim.

Front-line health and community care workers may therefore become accidentally exposed to fentanyl when working with a patient who has used or overdosed on the drug or its analogs, or when conducting searches of a patient who has the drug with them. Some examples of tasks that could be hazardous and would require risk assessment include removing the clothing, searching, reviving, and transferring of a patient who has used the drug or has the drug in their possession. In addition, fentanyl may be transported on clothing to another location, thus exposing non-front line populations, such as the families or colleagues of the front line worker.

Fentanyl and its analogs can enter the body by inhalation, ingestion, or intravenous or intramuscular injection. Skin contact is also thought to be a potential exposure route, but is not likely to lead to overdose unless there is prolonged exposure to large volumes of highly concentrated fentanyl in powder form. Brief skin contact with fentanyl or its analogs is not expected to lead to toxic effects if any visible contamination is immediately removed. Overall, inhalation and incidental ingestion are the greatest threats to health and community care workers.

To protect against accidental exposure, current best practices require that standard personal protective equipment be worn by health and community care workers who are working with patients suspected or known to have been exposed to fentanyl or carfentanil. This may include gowns, aprons, eye protection, booties and (double) nitrile gloves based on the situational risk assessment conducted (including the form and quantity of the drug). Fit-tested N95 respirators are recommended to be worn if there is a risk of respiratory exposure to the drug.

Although naloxone is a safe and effective medication used to temporarily block the effects of fentanyl and other opioids, due to the high potency of fentanyl and its analogs, multiple doses of naloxone may be needed to treat a fentanyl overdose. Furthermore, naloxone only temporarily blocks the effects of respiratory depression caused by opioids (for 30-90 minutes) so medical attention is still required following its administration. As such, while naloxone can be employed to treat first responders who have been inadvertently exposed to fentanyl or other opioids, it would be preferable to avoid this exposure in the first place.

Another example of first responders subject to exposure to dangerous substances on an ongoing basis are firefighters. A study from the University of Ottawa has found that firefighters absorb toxic chemicals from smoke through their skin while on the job. Keir et al. “Elevated Exposures to Polycyclic Aromatic Hydrocarbons and Other Organic Mutagens in Ottawa Firefighters Participating in Emergency, On-Shift Fire Suppression”, Environ. Technol. 2017, 51, 21, 12745-12755. The report revealed that, after fighting fires, urine samples of OFS workers show four times the potential for DNA damage and contain between three and five times more polycyclic aromatic hydrocarbons (PAH), which are harmful chemical compounds often linked to cancer. Examples of common PAH metabolites found in firefighters include naphthalene, pyrene, phenanthrene and fluorine. Skin swabs of firefighters in the study found that they were exposed to these chemicals through dermal exposure to combustion products.

Particulates generated from burning wood or other organic matter are composed of chemically inert carbon particles that become adsorbed (coated) with harmful chemical compounds. Firefighters are exposed to PAH during a fire, and a coating of PAHs can remain on portions of their bodies for days afterwards. Current protocols employ the use of soap and water, which can be inconvenient in the field where no shower-type facilities are available. Other alternatives involve the use of alcohol based antiseptic wipes; however, these may not be highly effective at removing hydrophobic PAHs from the skin.

Therefore there is a need for low-cost, non-toxic, convenient and readily accessible products capable of minimizing exposure of first responders to noxious substances in the field.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide polyol fatty ester compositions and products for minimizing exposure to a noxious substance. In accordance with an aspect of the present invention, there is provided a method for minimizing exposure to a noxious substance, the method comprising the step of administering a spray composition onto a surface contaminated by the noxious substance, wherein the spray composition comprises a polyol fatty ester.

In accordance with another aspect of the present invention, there is provided a spray composition to minimize exposure to a noxious substance, wherein the spray composition comprises a polyol fatty ester.

In accordance with another aspect of the present invention, there is provided use of the spray composition to minimize exposure to a noxious substance.

In accordance with another aspect of the present invention, there is provided a method for minimizing exposure to a noxious substance, the method comprising the step of wiping a surface contaminated with the noxious substance with a polyol fatty ester wipe, wherein the wipe comprises a polyol fatty ester coated on or impregnated in a flexible sheet material.

In accordance with another aspect of the present invention, there is provided a polyol fatty ester wipe for minimizing exposure to a noxious substance, wherein the wipe comprises a polyol fatty ester coated on or impregnated in a flexible sheet material

In accordance with another aspect of the present invention, there is provided use of the polyol fatty ester wipe to minimize exposure to a noxious substance.

### DETAILED DESCRIPTION OF THE INVENTION

The terms “polyol fatty ester” and “polyol fatty polyester” are used interchangeably in the present disclosure to describe a polyol molecule that has been esterified with fatty acid residues on multiple hydroxyl groups. In a preferred embodiment, the polyol is a sugar molecule or a sugar alcohol molecule.

The terms “sugar fatty ester” and “sugar fatty polyester” are used interchangeably in the present disclosure to describe a sugar or sugar alcohol molecule that has been esterified with fatty acid residues on multiple hydroxyl groups.

As used herein, the term “about” refers to a +/−10% variation from the nominal value. It is to be understood that such a variation is always included in a given value provided herein, whether or not it is specifically referred to.

Unless defined otherwise, all technical and scientific terms used herein have the same meaning as commonly understood by one of ordinary skill in the art to which this invention belongs.

In one embodiment, there is provided a spray composition comprising a polyol fatty ester suitable for use in minimizing exposure to a noxious substance. In accordance with one embodiment of the present invention, the polyol fatty ester spray composition is dispensed using a spray dispenser, in a similar manner to the dispensing of mace or a gas propelled sunscreen, such that the spray is applied to and coats a surface that has been identified as possibly being contaminated with a noxious substance.

The polyol fatty ester spray is suitable for use in sequestering any noxious substance present on a contaminated surface. For example, if a first responder identifies an overdose victim, and suspects that they may have fentanyl contamination, they would apply the polyol fatty ester spray onto the potential contamination points: the victim's hands, face, table tops, etc. Any fentanyl contacted by the polyol fatty ester spray would be sequestered and rendered inert, leading to a greatly reduced contamination risk for the first responder.

In another embodiment, there is provided a wipe-type sheet material product comprising a polyol fatty ester suitable for use in minimizing exposure to a noxious substance. In accordance with one embodiment of the present invention, the polyol fatty ester is provided as a coating on the sheet material. In accordance with another embodiment, the polyol fatty ester is impregnated in the sheet material. In accordance with the present invention, the polyol fatty ester sheet material is used to wipe a surface that has been identified as possibly being contaminated with a noxious substance.

The polyol fatty ester wipe is suitable for use in removing any noxious substance from a contaminated surface. For example, the wipes may be used in the field for the cleaning and removal of toxins and contaminants such as soot, smoke and other potential carcinogens from the skin of a firefighter or any other surface that has been contaminated by exposure to combustion products. Any toxins and contaminants contacted by the polyol fatty ester on the wipe would be removed, sequestered and rendered inert, leading to a greatly reduced contamination risk for the first responder. The immediate removal of the combustion residues also minimizes absorption through the skin, thus reducing the overall exposure of the first responder to the noxious substances. The polyol fatty ester wipes therefore provide a convenient and readily accessible means for the immediate removal of combustion residues or other noxious substances from the skin and other surfaces, thus avoiding the need for specialized equipment in the field.

The polyol fatty esters suitable for use in the sprays and wipes of the present invention are preferably selected from sugar fatty esters, sugar alcohol fatty esters, or mixtures thereof.

The term “sugar” is used herein in its conventional sense as generic to mono-, di-, and trisaccharides. The term “sugar alcohol” is also used in its conventional sense as generic to the reduction product of sugars wherein the aldehyde or ketone group has been reduced to an alcohol. To be suitable for use in the present invention, the sugar or sugar alcohol must contain at least four hydroxyl groups. The polyol fatty ester compounds are prepared by reacting mono-, di- or trisaccharides or sugar alcohols with fatty acids as discussed below.

Examples of suitable monosaccharides include those containing four hydroxyl groups such as xylose, arabinose, and ribose. Sugar alcohols having four hydroxyl groups, i.e., xylitol and erythritol, are also suitable. Monosaccharides containing five hydroxyl groups, such as glucose, mannose, galactose, fructose, sorbose, are also suitable. A sugar alcohol derived from glucose, mannose, or sorbose, e.g., glucitol, mannitol, or sorbitol, contains six hydroxyl groups and is also suitable as the alcohol moiety of the fatty acid ester compound. Examples of suitable disaccharides are maltose, lactose, and sucrose, all of which contain eight hydroxyl groups. Examples of suitable trisaccharides are maltotriose and raffinose.

The sugar fatty esters of the present invention are sugars or sugar alcohols that have been esterified with at least four fatty acid groups, preferably six, seven or eight fatty acid groups.

In preparing polyol fatty esters of the present invention, a sugar or sugar alcohol compound such as those identified above are esterified with fatty acids having from about 6 to about 24 carbon atoms, preferably from 8 to 22 carbon atoms, more preferably from 12 to 24 carbon atoms. The fatty acids can be derived from suitable naturally occurring or synthetic fatty acids and can be saturated or unsaturated, including positional and geometric isomers (e.g., cis and trans isomers).

In one embodiment, the polyol is esterified with fatty acids having 12 or more carbon atoms. Examples of such fatty acids having 12 or more carbon atoms include lauric, myristic, myristoleic, palmitic, palmitoleic, stearic, oleic, linoleic, linolenic, eleostearic, arachidic, arachidonic, behenic, and erucic acid.

In one embodiment, at least about 90% of the fatty acids are selected from the group consisting of palmitic, stearic, oleic, linoleic, behenic, and mixtures thereof. In one embodiment, at least about 80% of the fatty acids must contain not less than 12 carbon atoms. In one embodiment, at least about 95% of the fatty acids will contain not less than 12 carbon atoms. In one embodiment, the polyol fatty esters employed are those wherein at least about 90% of the fatty acids contain not less than 16 carbon atoms.

In one embodiment, the polyol fatty ester is a sucrose fatty ester. Preferred sucrose fatty esters have the majority of their hydroxyl groups esterified with fatty acids. In one embodiment, at least about 85%, and more preferably at least about 95%, of the sucrose fatty esters are selected from the group consisting of octaesters, heptaesters and hexaesters, and mixtures thereof. Preferably, no more than about 35% of the esters are hexaesters or heptaesters, and at least about 60% of the esters are octaesters. Most preferably, at least about 70% of the esters are octaesters. It is also most preferred that the polyol fatty esters have a total content of penta- and lower esters of not more than about 3%.

In a preferred embodiment, the sucrose fatty ester is a sucrose molecule that has been esterified on at least 6 of the free hydroxyl groups.

Sucrose fatty esters useful in the spray compositions and wipes disclosed herein respectively have a fatty chain length of, preferably from about C12 to about C30, more preferably from about C14 to about C26, still more preferably from about C16 to about C22. In the present invention, “sucrose fatty esters having a fatty chain length” means that: at least about 50%, preferably at least about 60%, more preferably at least about 70%, still more preferably at least about 80% of the fatty chains of the sucrose fatty esters have the fatty chain length which is specified above.

The polyol fatty esters suitable for use herein can be prepared by a variety of methods known to those skilled in the art. These methods include: transesterification of the polyol with methyl, ethyl or glycerol fatty acid esters using a variety of catalysts; acylation of the polyol with a fatty acid chloride; acylation of the polyol with a fatty acid anhydride; and acylation of the polyol with a fatty acid. Exemplary processes for the preparation of sucrose fatty esters are described in U.S. Pat. Nos. 2,831,854, 3,600,186, 3,963,699, 4,517,360 and 4,518,772 (all herein incorporated by reference).

In the context of the present invention, the polyol fatty ester is a substance that is highly hydrophobic and is not capable of being internalized or absorbed through the human epithelium. One non-limiting example of a sucrose fatty ester is Olestra®. However, other sugar polyesters capable of interacting with the noxious substance and preventing that substance from being absorbed through the epithelium are also within the scope of the present invention. Olestra comprises a sucrose disaccharide esterified with six to eight long fatty acid chains. It has been suggested that steric hindrance due to the long fatty acid chains prevents hydrolysis of the esters by enzymes, and it is believed that this inability of Olestra to be metabolized prevents its absorption though the epithelial cells of the digestive tract. Olestra also interferes with the absorption of other lipophilic molecules, such as fat-soluble vitamins due to the partition of these molecules into the non-absorbable olestra, which then carries the molecules out of the body as described in Lawson et al. in “Olestra, a nonabsorbed, noncaloric replacement for dietary fat: a review.” Drug metabolism reviews 29.3 (1997): 651-703. In fact, when Olestra was approved for human consumption, it was specified that food containing Olestra had to be fortified with fat soluble vitamins to replace the vitamins that were stripped from the body of the consumer.

Without being bound by theory, it is the ability of the sucrose fatty ester to sequester hydrophobic molecules that forms the basis for the usefulness of the present polyol fatty ester spray and wipes in minimizing exposure to noxious hydrophobic substances.

Examples of noxious hydrophobic substances that can be sequestered using the polyol fatty ester spray or removed using the polyol fatty ester wipes of the present invention include, but are not limited to, most common street drugs, including opioids such as fentanyl, carfentanil and other fentanyl derivatives, codeine, morphine, heroin, methadone and butorphanol; cocaine and its derivatives, such as crack cocaine; amphetamines, such as methamphetamine or MDMA (ecstasy); flunitrazepam (rohypnol, or the “date rape drug”); gamma hydroxybutyrate (GHB); ketamine; PCP; dextromethorphan (DXM); and LSD.

Examples of noxious hydrophobic substances that can be removed using the polyol fatty ester coated or impregnated sheet material wipes of the present invention include ash, soot, polycyclic aromatic hydrocarbons (PAHs) for example, naphthalene, pyrene, and phenanthrene, fluorine, benzene, chloroform, formaldehyde, and chlorinated hydrocarbons. It is also within the scope of the invention that the polyol fatty ester wipes can be used by first responders in any other where a first responder may be exposed to a noxious substance, including exposure to street drugs as described above, or exposure to the active oils of poison oak or poison ivy and the like.

Additional examples of commercial sucrose polyesters suitable for in the sprays and wipes of the present invention include, for example, the following sucrose polyesters available from P&G Chemicals: Sefose 1618H, having a melting point of about 60° C., esterification degree of about 8, an alkyl distribution containing about 87% of C18 and about 13% of C16; Sefose 2275C, having a melting point of about 70° C., esterification degree of about 8, and an alkyl distribution containing about 80% of C22 and about 20% of C18; Sefose 1618U, having a melting point of about −5° C., esterification degree of about 8, and an alkyl distribution containing about 86% of C18 and about 12% of C16 and about 2% of others; and Sefose 1618S, having a melting point of about 10° C., esterification degree of about 6, and an alkyl distribution containing about 87% of C18 and about 13% of C16.

In one embodiment, the polyol fatty ester is liquid at room temperature. In one embodiment, the polyol fatty ester has a melting point of less than around 35° C. In one embodiment, the polyol fatty ester has a melting point of less than around 30° C. In one embodiment, the polyol fatty ester has a melting point of less than around 25° C.

Where the polyol fatty ester is liquid at room temperature, the spray composition may be formulated without solvents or carriers, and may be applied using a suitable spray mechanism.

In embodiments wherein the polyol fatty ester is solid at room temperature, the spray composition further comprises a suitable solvent to provide a solution of the polyol fatty ester suitable for spray application. Suitable solvents include, but are not limited to, hydrocarbons such as hexane and pentane, alcohols such as ethanol or iso-propanol, glycerin, or vegetable oils such as soybean oil, canola oil or safflower oil.

In one embodiment, the spray composition comprises from about 10% to about 90% polyol fatty ester by weight of the total composition, and a suitable solvent. In one embodiment, the spray composition comprises at least about 20% polyol fatty ester by weight of the total composition, and a suitable solvent. In one embodiment, the spray composition comprises at least about 40% polyol fatty ester by weight of the total composition, and a suitable solvent. In one embodiment, the spray composition comprises at least about 60% polyol fatty ester by weight of the total composition, and a suitable solvent.

In accordance with one embodiment of the present invention, the spray composition may optionally comprise one or more additional ingredients as appropriate. For example, a dye or other coloring agent may be added to assist in visualizing the extent of the spray application to ensure complete coverage.

It is within the scope of the present invention that the spray composition may further optionally comprise additional ingredients including one or more of: preservatives, viscosity modifiers, indicator compounds that indicate the presence of the noxious substance,

Spraying can be done by atomizing the spray composition with a stream of air, with an aerosol, or using hydraulic techniques. Spraying can be in the form of a fine mist or spray or in coarse droplets. Various types of spray devices and spray nozzles (atomizers) are described in Perry & Chilton, Chemical Engineers' Handbook, 5th Edition, pp. 18-49 to 18-50 and 18-61 to 18-64, McGraw-Hill, New York (1973). The spray nozzles include hollow core, solid core, oval-orifice fan, deflector jet, impinging jet, by-pass, and poppet. The spray composition can be sprayed on the target surface in a single application, or in multiple applications as required to form a continuous coating.

In one embodiment, the spray composition is administered using an aerosol spray dispensing system. Using an aerosol spray type of administration, an aerosol mist of the spray composition is dispensed using a can or bottle that contains the composition and propellant under pressure. The propellant may be any propellant as is known in the art, including but not limited to, a mixture of volatile hydrocarbons (including but not limited to propane, n-butane and isobutene), dimethyl ether (DME), methyl ethyl ether, nitrous oxide, or carbon dioxide.

The spray compositions of the present invention can be contained or dispensed in any known or otherwise effective aerosol container or delivery system. All such containers or delivery systems should be compatible with the essential and any selected optional ingredients of the spray composition of the present invention. Other suitable aerosol dispensers include those containing compressed air propellant(s) which can be filled into the dispenser by means of a pump or equivalent device prior to use.

In those embodiments employing aerosol spray dispensing of a solution of the polyol fatty ester, an aerosol concentrate of the polyol fatty ester may be prepared by adding the solvent to the polyol fatty ester in an appropriately sized container and mixing until the polyol fatty ester is completely solubilized. Any remaining ingredients (except propellants) are added allowing each to be fully incorporated before adding the next ingredient. The concentrate is then filled into the aerosol dispenser, and then propellant is added using any of the methods commonly accepted in the aerosol industry.

In one embodiment, the spray composition is administered using a pump spray dispensing system. Depending on the sprayer, the nozzle may or may not be adjustable, so as to select between squirting a stream, aerosolizing a mist, or dispensing a spray. Spray bottles with trigger-style actuators may be suitable for the present application.

In one embodiment, the polyol fatty ester is provided as a coating on a flexible sheet material, and the coated sheet material is used to wipe a surface that has been identified as possibly being contaminated with a noxious substance.

In accordance with the present invention, the wipe is formed from a sheet material that may be any suitable flexible fibrous or non-fibrous material that can be coated or impregnated with the polyol fatty ester. In one embodiment, the sheet material is a woven fabric. In one embodiment, the sheet material is a nonwoven fibrous web.

As used herein, the term “nonwoven web” refers to a web having a structure of individual fibers or threads which are interlaid, but not in a repeating pattern as in a woven or knitted fabric, which do not have randomly oriented fibers. Nonwoven webs or fabrics can be formed using many processes known in the art, including meltblowing processes, spunbonding processes, hydroentangling processes, spunlacing processes, airlaying, and bonded carded web processes.

As used herein, the term “spunbonding ” refers to a process for forming small diameter fibers by extruding molten thermoplastic material as filaments from a plurality of fine, usually circular capillaries of a spinneret with the diameter of the extruded filaments then being rapidly reduced. Spunbond fibers are generally not tacky when they are deposited on a collecting surface.

As used herein, the term “meltblowing” refers to a process in which fibers are formed by extruding a molten thermoplastic material through a plurality of fine, usually circular, die capillaries as molten threads or filaments into converging high velocity, usually heated, gas (for example air) streams which attenuate the filaments of molten thermoplastic material to reduce their diameter, which may be to microfiber diameter. Thereafter, the meltblown fibers are carried by the high velocity gas stream and are deposited on a collecting surface, often while still tacky, to form a bed of randomly dispersed meltblown fibers.

The fibers produced by these processes may then be further processed into a web by any number of techniques known in the art including, but not limited to: entangling (including hydroentangling), felting (or needling), or other non-melt bonding techniques, or combinations thereof. In one embodiment, the fibers are entangled. In one embodiment, the fibers are melt-bonded or thermal bonded.

The nonwoven web may also comprise one ply or layer, or a plurality of plies. The fibrous web may therefore be a single layer of material or may be constructed of multiple layers superposed upon one another. If the fibrous web is composed of multiple layers, the layers may be bonded together by heat sealing, needling, or any number of means known in the art.

The sheet material may also include a line of weakness, including, but not limited to, a line of perforations, laser scores, or tear-initiating notches, which would facilitate the use of a portion or part of the sheet material.

In one embodiment, the sheet material is provided as a textured web, having a textured surface that provides additional scrubbing effect to facilitate removal of the contaminants from the surface to be cleaned.

In one embodiment, the wipes are provided with the polyol fatty ester coated on one side of the sheet material. In one embodiment, the wipes are provided with the polyol fatty ester coated on both sides of the sheet material. In one embodiment, the wipes are provided with the polyol fatty ester impregnated throughout the sheet material.

In one embodiment, the polyol fatty ester is coated on the nonwoven fibrous web after it has been formed into nonwoven web sheet material. In one embodiment, the polyol fatty ester is coated onto the fibers prior to the fibers being formed into the nonwoven fibrous web.

The constituent fibers of nonwoven web can be comprised of polymers such as polypropylene (PP), polyethylene (PE), poly-4-methylpentene (PMP), polyester, polyethylene terephthalate (PET), and blends thereof; cellulose, rayon, cotton, wool, or other natural materials; or blends of polymers and natural materials. The fibers can also comprise a super absorbent material such as polyacrylate or any combination of suitable materials.

In one embodiment, the polyol fatty ester wipes also comprise additional ingredients such as natural and/or synthetic surfactants, humectants, moisturizers, natural extracts, and/or chelating agents.

In one embodiment, the polyol fatty ester wipes are packaged as individually wrapped, ready-to-use wipes. In one embodiment, the polyol fatty ester wipes are packaged as a roll of tear-apart sheets.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1-10. (canceled)

11. A spray composition for minimizing exposure to a noxious substance, wherein the spray composition comprises a polyol fatty ester.

12. The composition of claim 11, wherein the polyol fatty ester is a sugar fatty ester.

13. The composition of claim 12, wherein the sugar fatty ester is a sugar or sugar alcohol that is esterified with four or more fatty esters.

14. The composition of claim 12, wherein the sugar fatty ester is sucrose fatty ester that is esterified with six or more fatty esters.

15. The composition of claim 11, wherein the fatty esters are derived from lauric acid, myristic acid, myristoleic acid, palmitic acid, palmitoleic acid, stearic acid, oleic acid, linoleic acid, linolenic acid, eleostearic acid, arachidic acid, arachidonic acid, behenic acid, erucic acid, or combinations thereof.

16. The composition of claim 11, wherein the spray composition comprises a solvent.

17. The composition of claim 16, wherein the solvent is selected from hydrocarbons, alcohols, glycerin, a vegetable oil, or combination thereof.

18. The composition of claim 11, wherein the noxious substance is fentanyl, carfentanil, or a combination thereof.

19. The composition of claim 11, wherein the spray composition is for administration using a pump spray.

20. The composition of claim 11, wherein the spray composition is for administration using an aerosol spray.

21-29. (canceled)

30. A polyol fatty ester wipe for minimizing exposure to a noxious substance, wherein the wipe comprises a polyol fatty ester coated on or impregnated with a flexible sheet material.

31. The wipe of claim 30, wherein the polyol fatty ester is a sugar fatty ester.

32. The wipe of claim 31, wherein the sugar fatty ester is a sugar or sugar alcohol that is esterified with four or more fatty esters.

33. The wipe of claim 32, wherein the sugar fatty ester is sucrose fatty ester that is esterified with six or more fatty esters.

34. The wipe of claim 30, wherein the fatty esters are derived from lauric acid, myristic acid, myristoleic acid, palmitic acid, palmitoleic acid, stearic acid, oleic acid, linoleic acid, linolenic acid, eleostearic acid, arachidic acid, arachidonic acid, behenic acid, erucic acid, or combinations thereof.

35. The wipe of claim 30, wherein the flexible sheet material is a nonwoven fibrous web.

36. The wipe of claim 35, wherein the non-woven fibrous web is formed from fibers comprising polymers selected from polypropylene (PP), polyethylene (PE), poly-4-methylpentene (PMP), polyester, polyethylene terephthalate (PET), and combinations thereof; natural materials selected from cellulose, rayon, cotton, wool or other natural materials; or blends of polymers and natural materials.

37. The wipe of claim 30, wherein the flexible sheet material is a woven fabric.

38. (canceled)

39. Method for minimizing exposure to a noxious substance, the method comprising the step of administering a spray composition onto a surface contaminated by the noxious substance, wherein the spray composition is as defined in claim 11, wherein the spray composition is administered using a pump spray or an aerosol spray.

40. Method for minimizing exposure to a noxious substance, the method comprising the step of wiping a surface contaminated with the noxious substance with a polyol fatty ester wipe, wherein the wipe is as defined in claim 30.
